(19) United States
(12) Patent Application Publication
(10) Pub. No.: US 2021/0354381 A1
(43) Pub. Date: Nov. 18, 2021

(54) ROBOTIC BIOPRINTING SYSTEM

(71) Applicant: Poietis, Pessac (FR)

(72) Inventors: Bertrand Viellerobe, Merignac (FR); Fabien Guillemot, Preignac (FR); Evarzeg Le Bouffant, Le Haillan (FR)

(21) Appl. No.: 17/288,467

(22) PCT Filed: Oct. 24, 2019
(86) PCT No.: PCT/FR2019/052542

§ 371 Date: Apr. 23, 2021

(30) Foreign Application Priority Data
Oct. 25, 2018 (FR) 1859891

(51) Int. Cl. B29C 64/227; B33Y 10/00; B33Y 30/00

(52) U.S. Cl. CPC B29C 64/227; B29C 64/268; B33Y 30/00; B33Y 10/00

(57) ABSTRACT

A bioprinting system for manufacturing a structured biological material, from materials of which at least a portion is constituted by biological particles (cells and cell derivatives), comprises: a) a printing assembly containing at least one print head for printing objects of biological interest and at least one target, b) a supply source for supplying the print head with objects of biological interest, c) a means for bioprinting the objects of biological interest, and d) a means for moving the print head relative to the target, characterized in that the movement means is constituted by a robot controlling the movement of the target along six axes, at least one of the print heads being stationary during the printing phase.

### CROSS-REFERENCE TO RELATED APPLICATIONS

This application is a national phase entry under 35 U.S.C. § 371 of International Patent Application PCT/FR2019/052542, filed Oct. 24, 2019, designating the United States of America and published as International Patent Publication WO 2020/084263 A1 on Apr. 30, 2020, which claims the benefit under Article 8 of the Patent Cooperation Treaty to French Patent Application Serial No. 1859891, filed Oct. 25, 2018.

### TECHNICAL FIELD

The present disclosure relates to the field of additive manufacturing, which makes it possible to artificially produce biological tissues, referred to as “bioprinting.” Bioprinting allows the spatial structuring of living cells and other biological products, biomaterials, biochemicals, or biocompatibles by positioning them sequentially through layer-by-layer deposition under the control of a computer in order to develop living tissues and organs for tissue engineering, regenerative medicine, pharmacokinetics, and more generally for biological research.

### BACKGROUND

The primary use of bioprinting relates to the preparation of synthetic living tissues for experimental research, replacing tissues taken from living beings-both animals and humans-in order to avoid regulatory and ethical problems. In the longer term, bioprinting will allow organs to be produced for transplantation without the risk of rejection, including the epidermis, bone tissue, parts of the kidney, the liver, as well as on other vital organs, heart valves, or hollow structures such as vascular structures.

The manufacture of a tissue by 3D bioprinting can be broken down into three sequential technological steps:
A pretreatment for the designing of a digital model that will define how the differentiated cells or stem cells will be prepared in culture for the constitution of the bio-ink and then printed layer by layer.
Automated printing of the tissue by the printer using various technologies (laser printing, biological ink jet, micro-extrusion, etc.).
Maturation of printed tissues, during which the assembled cells and biomaterials evolve and interact together to form a functional and viable tissue.

The general principle consists in preparing a source containing a biological ink incorporating the various elements that can be transferred by a controlled energy supply emanating from an activation source, for example, a laser, an electromechanical or a sound pulse, or even a projection, in the direction of a receiving target on which the transferred elements form a two- or three-dimensional matrix by additive printing. The arrival position on the target of each transferred element is determined by the relative positioning of the source with respect to the target. Generally, the activation source is guided on the XY plane perpendicular to the direction of transfer in order to determine the position of each element on the target.

The present disclosure relates more particularly to the displacement of objects of biological interest from the printing source relative to the target and, more particularly, to robot-aided displacement.

It is the object of the present disclosure to transfer objects of biological interest comprising living cells (for example, pluripotent stem cells or any other differentiated cells), sometimes of different types, as well as biological products such as collagen and, more generally, extracellular matrix materials, from a source to a target.

The objects of biological interest can be brought together in a fluid to form a “bio-ink” containing biological particles such as living cells, for example. These bio-inks are then prepared and packaged in sterile form so that they can be used to print biological tissue when the time comes.

Within the meaning of the present patent, bioprinting refers to the spatial structuring of living cells and other biological products by means of a method that creates a geometric structure, particularly a stack of layers formed by individualized deposits of objects of biological interest, with the aid of a computer in order to develop living tissues and organs for tissue engineering, for regenerative medicine, pharmacokinetics, and more generally for biological research. Bioprinting involves the simultaneous deposition of living cells and biomaterials layer by layer to make living tissues such as artificial structures of the skin, heart valves, cartilage, heart tissue, kidneys, liver, as well as other vital organs or hollow structures such as the bladder as well as vascular structures.

One example of a device for printing biological elements by laser based on the technique called “Laser-Induced Forward Transfer” (LIFT) is described in European patent EP3234102. It comprises a pulsed laser source emitting a laser beam, a system for focusing and orienting the laser beam, a donor medium comprising at least one biological ink, and a recipient substrate positioned so as to receive the material emitted from the donor medium.

The laser beam impacts the donor support while being oriented in an approximately vertical direction and in a direction from top to bottom, i.e., in the same direction as the gravitational force. The biological ink is thus placed under the slide so as to be oriented downward toward the recipient substrate that is placed under the donor medium.

The known prior art includes patent application US2016/068793, which describes a manufacturing assembly comprising a sterilizable chamber containing at least one three-dimensional printing device (additive manufacturing), a computer numerical control (CNC) finishing head (subtractive manufacturing), a vacuum-forming unit, an injection-molding unit) and a laser cutting unit, an ultrasonic welding unit, as well as an Arman robotic analysis device, a sampling device, or a combination thereof.

A plurality of individual sterilizable chambers can be aseptically connected to an array of sterilizable chambers, which provides additional functionality for the manufacturing assembly.

This solution is not intended for biological printing and uses a heating head for coating active pharmaceutical ingredients.

It is in no way intended for the deposition of objects of biological interest comprising living cells and intercellular materials to form living tissue.

Patent application WO2018072265 describes a 3D printing system based on coordinated multi-axis control and artificial vision measurement comprising a machine frame, a work bench for use in placing an artificial bone support, a printing device disposed above the work bench, a material transport device for use in transporting printing materials, image capture devices, a drive mechanism for adjusting the orientation of the printing device, and a control system; the printing device, the material conveying device, the image capturing devices, and the driving mechanism are all connected to the control system, the work bench is a parallel platform with six degrees of freedom that is connected to the machine frame, the driving mechanism is a six-axis robotic arm, and the printing device is connected to the six-axis robotic arm. During use, the artificial bone support is placed on the parallel platform with six degrees of freedom, the position of the printing device is controlled by means of the six-axis robotic arm, and precise spatial position control of a printing nozzle of the printing device is achieved through cooperation between the parallel platform with six degrees of freedom and the robotic arm with six axes, thereby obtaining three-dimensional pattern printing on complex and fine artificial bone surfaces and internal surfaces having a porous structure.

This document relates to the manufacture of artificial bone by means of 3D printing and in no way to the manufacture of a structured biological material from materials consisting at least partially of biological particles (living cells and cellular derivatives).

Patent application US2018141174 describes a machine tool that allows machining through removal and additional machining of a workpiece. The machine tool comprises a first spindle holder and a second spindle holder arranged in a first machining zone and intended to hold a workpiece, a lower cutting device holder and a tool spindle disposed in the first machining zone and intended to support a tool to allow material-removing machining of a workpiece, an additional machining head disposed in a second machining zone, and a robot arm intended to hold the workpiece and transport the workpiece between the first machining zone and the second machining zone. The additional machining head discharges material onto the workpiece held by the robot arm during additional machining of the workpiece. A machine tool that enables material-removing machining and additional machining of a workpiece is provided by such a configuration using a simple configuration.

This document does not relate to bioprinting.

Patent application US2010206224 describes a device for depositing layers, comprising:
a frame provided with an enclosure, the frame further supporting:
   a table that is intended to support an object to be manufactured and provided with a movable plate and first displacement means, “a material dispenser intended to place the material on the table in order to form the object and provided with second displacement means for at least one container, at least one nozzle, and at least one extrusion member,
   compacting means, and a control member that is intended to control the deposition of material on the table; at least the plate and the end of the nozzle are arranged inside the enclosure, and at least the means for moving the table and the dispenser and the control member are arranged outside the enclosure.

Firstly, the solutions of the prior art generally relate to additive printing solutions for inert materials and not to bioprinting, resulting in particular constraints involving the live nature of some of the transferred objects (living cells) and the need for precise positioning in order to take into account the subsequent progression during cell growth and decline and the structure of the biological tissue to be produced.

In the solutions of the prior art, the target is stationary during the printing phase, and the object printhead (or “donor”) is displaced on the activation axis passing through the target point on the target in order to position the elements to be transferred. This solution has several drawbacks. To wit, the displacement of the donor causes hydrodynamic disturbances of the carrier fluid in which the elements to be transferred are generally suspended, particularly in laser printing. These disturbances induce errors in positioning, targeting of objects, and ultimately the reproducibility of printing conditions. This constitutes a major limitation of existing solutions, particularly when it is desired to print at high resolution with the necessary reproducibility.

Furthermore, this solution is not optimized for nonplanar targets, e.g., a target that is intended for the bioprinting of a heart or vascular valve.

Finally, it is necessary to provide a plurality of means for moving the donor in order to put it in place before the printing phase or to remove it after the printing phase (or to carry out the placement and removal manually). The term “printing phase” is understood to mean the period during which the donor is subjected to a repetition of activations between the start of bioprinting and the end of a sequence of donor activation pulses.

### BRIEF SUMMARY

The present disclosure relates in its most general sense to a bioprinting system for the manufacture of a structured biological material from materials at least part of which consists of living biological particles (cells and cellular derivatives) according to claim 1.

In fact, in implementing the solution constituting the subject matter of the present disclosure, the (donor) printheads remain stationary during the printing step regardless of the technology used (laser, by nozzle, acoustics, etc.). It is thus easy to maintain fixed and optimal printing parameters since the printing conditions remain identical at all points of the printing field. The robot arm also handles the positioning in terms of the distance between the target and the head: the donor-recipient distance.

This must be known and maintained during the printing phase, because it constitutes one of the parameters that strongly influences the shape and the quantity of the material that is deposited on the target substrate.

The immobile nature of the printheads also makes it possible to equip the heads with characterization means (imaging, distance measurements, sensors, etc.), since they are linked to the frame of the bioprinter with sufficient space to integrate these measuring means without the constraint of having to move them like in the prior art.

According to variants considered in isolation or in combination:
the robot is a robotic arm having six degrees of freedom, with three axes intended for positioning and three axes for orientation along at least 180° for each axis of rotation, making it possible to displace and orient the target in a given workspace, the course of the displacements being greater than the largest dimension of the target,
the robot is of the hexapod type,
the robot is of the delta type,
the robot is of the hexapod or delta type and comprises means for turning the target,
the target is linked to the robot by an effector,
the system comprises a support for receiving a plurality of targets, the robot controlling the extraction of a target for displacement relative to the bioprinting means,
the system comprises a second robot for an additional function (pipetting, etc.) in simultaneous operation,
the robot also handles the initial positioning of the donor and preparation thereof,
the bioprinting system incorporates at least one laser bioprinting means,
the bioprinting system incorporates at least one nozzle bioprinting technique,
the bioprinting system incorporates a combination of nozzle and laser bioprinting techniques.

The present disclosure also relates to a bioprinting method for the manufacture of a structured biological material from materials at least a part of which consists of biological particles (cells and cellular derivatives) consisting in controlling the displacement of at least one target by means of a robot in three dimensions relative to at least one stationary printhead during the printing phase.

Optionally, the method further comprises displacing the target relative to at least one additional workstation.

According to one variant, the displacement is controlled in order to maintain a constant distance between a target having a nonplanar surface, and a printhead.

### BRIEF DESCRIPTION OF THE DRAWINGS

The present disclosure will be better understood on reading the following description, which concerns a non-limiting exemplary embodiment that is illustrated by the accompanying drawings, in which:

FIG. 1 is a view along a sectional plane of an exemplary embodiment of the present disclosure,

FIGS. 2 to 5 are views of a robotic arm at different stages of handling the target,

FIG. 6 is a front view of one of the machine according to the present disclosure.

FIG. 7 shows an exemplary embodiment in the form of a pipettor associated with the Robot.

FIG. 8 shows a perspective view of a pneumatic and tube system associated with the robot.

### DETAILED DESCRIPTION

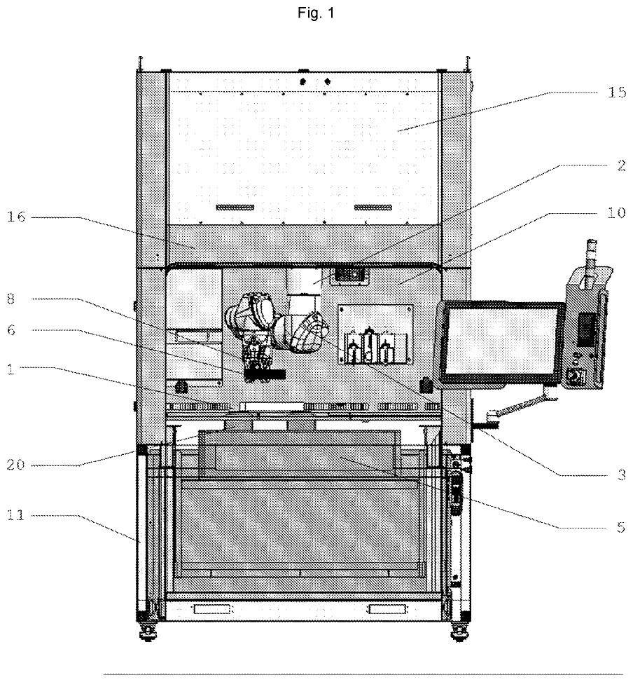

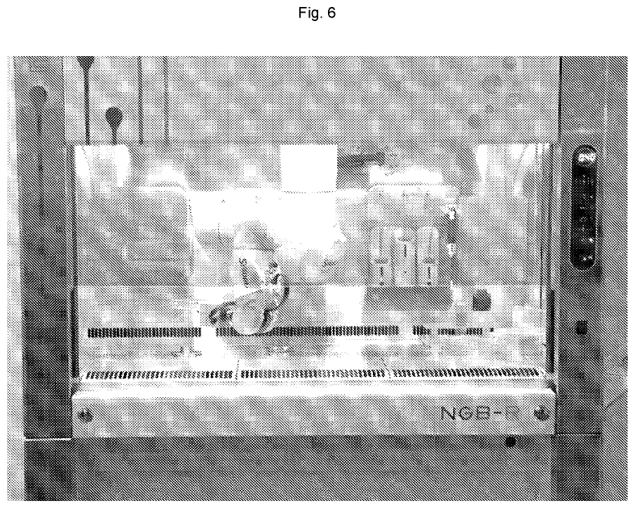

The bioprinter, of which FIG. 1 or FIG. 6 illustrate exemplary embodiments, consists of a frame whose lower part (11), which is non-sterilizable, contains the bioprinting means (5), for example, the optical head, the laser, and the imaging systems for a laser bioprinter.

This frame is covered by a clean (hood type) or sterilizable (isolator type) enclosure (10) consisting of a chamber with a laminar airflow ceiling ventilator (hood) or positive pressure ceiling supplied by a blower (isolator) (15) via a filter cartridge (16). A robotic arm (3) placed in this sterilizable chamber (10) handles the displacement of a target (6) relative to a printhead (1). An optionally sealed optical window (20) allows transmission of the laser beam and imaging beams between the sterilizable enclosure (10) and the printing medium (5) placed in a non-sterilizable zone.

The robotic arm (3) handles the displacement of the target (6) in the work zone during the printing phase, and outside of this work zone before the printing phase, in order to remove a target from a stock of blank targets, or into a maturation zone after the printing phase.

In the example described, the robot consists of an anthropomorphic robotic arm (3) having, in an inherently known manner, six axes of rotation. The robot shown in the illustrations is commercially available and was designed and manufactured by the company STAUBLI ROBOTICS. It has the particularity of existing in a sterilizable version that is compatible with good manufacturing practices in the pharmaceutical sector and is therefore compatible with the manufacture of clinical-grade tissues. It is secured by means of a foot (2) and comprises four segments and two elbows (4, 7). These different elements are assembled so as to be able to rotate relative to one another about the axes of rotation. The last segment (8) generally carries a working tool consisting of an effector in the form of a clamp (9) for gripping the target (6).

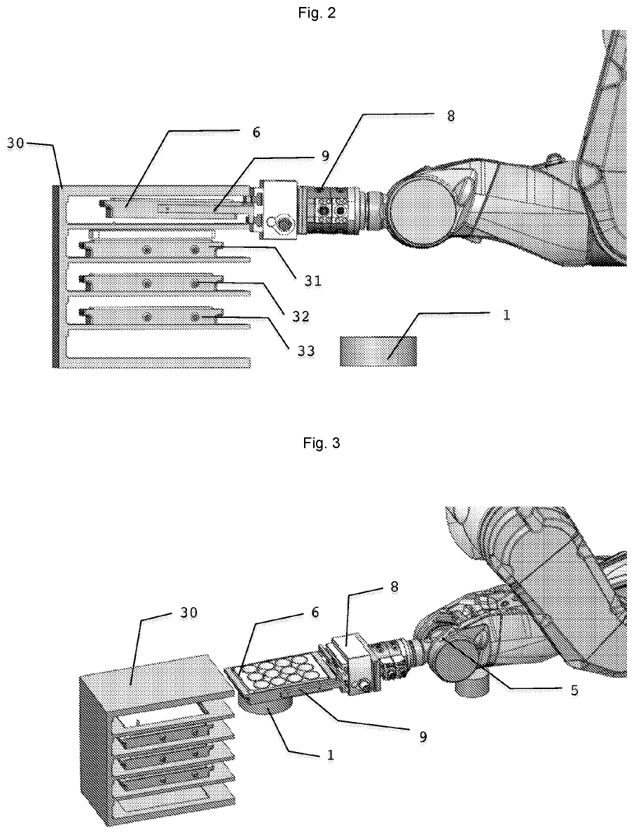

FIGS. 2 to 5 illustrate a succession of positions of the robotic arm (3) and of the target (6).

In the first situation illustrated by FIG. 2, a medium (30) is loaded with a plurality of blank targets (6, 31, 32, 33) that are ready to receive bioprinted elements. One of the targets (6) is extracted from the support (30) by the clamp (9) as shown in FIG. 3.

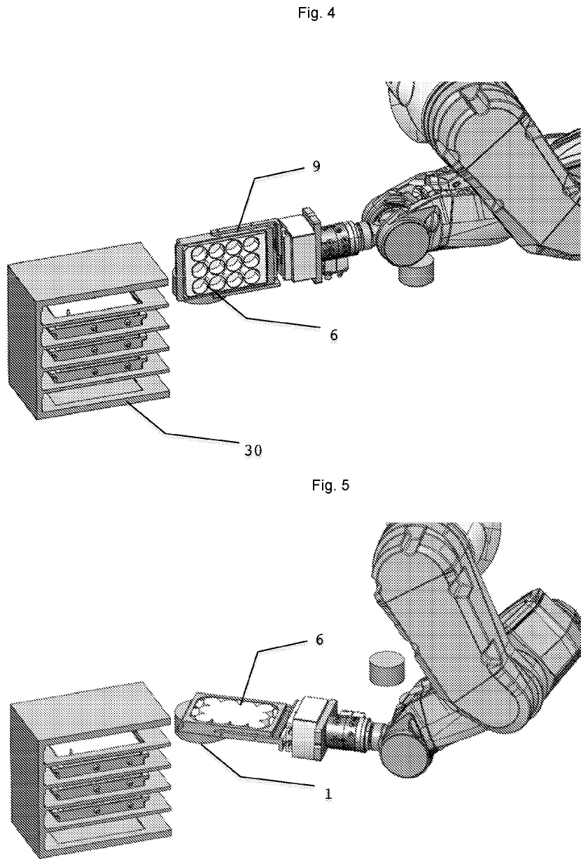

The target (6) can be turned over as shown in FIG. 4 by pivoting the clamp (9), for example, in order to print alternately on one side and on the other side.

The target (6) is then positioned above the donor (1) and moved on the XY plane, and possibly along the Z axis, in order to very precisely position the target (6) so that the projection of the element coming from the donor (1) arrives at the location provided by the modeling program for the tissue to be printed. For the majority of bioprinting technologies, the distance between the donor and the recipient substrate constitutes a very important parameter for print quality and reproducibility. Thus, the robot can maintain a stationary or regulated value of this distance at all times even if the substrate is not flat.

In this exemplary embodiment, the printing zone is isolated from the outside by an enclosure (10), which makes it possible to dissociate the power source (5) from the printing and handling zone of the receiving substrate where the robot is located. This is a major difference from the examples of the prior art in which the power source and the printing zone form a single entity. This dissociation provides a major advantage in terms of the protection and stability of the printing process.

The different robot positions described in this exemplary embodiment are sent to the robot via a SIEMENS®-type programmable logic controller, which makes it possible to perfectly schedule and synchronize all of the actions performed by the different printheads and the robot during bioprinting. The sequencing and synchronization of the various elements described here must be carried out unequivocally and over very short periods of time in order to ensure rapid printing and maintain the viability of the tissue being printed and the fidelity of what is printed relative to the starting digital model.

The path of the robot in this context corresponds to two types of operation:
positioning: this is the positioning of the recipient at different locations of the machine (reloading, imaging, printing, etc.). This refers to displacements for traveling from one area of the machine to another without searching for a specific path, except that it is secure in avoiding any collisions with the various elements that are present in the enclosure. The robot thus makes it possible to manage the multimodal aspect of a bioprinter when it is equipped with a plurality of different printing and characterization methods. The robot can also make it possible to position the target relative to the donor for laser printing at the desired distance. In some configurations, the robot will be able to switch from a high-resolution (HR) laser printhead to a low-resolution (BR) laser printhead.
the printing path: this is the creation of printing patterns. Indeed, for the printing methods by nozzle, the robot handles the printing path by movement X, Y (see Z) of the recipient. In this case, it should be emphasized that it is capable of working in two modes: the first “stop and shoot” mode corresponding to a path of discontinuous points, and the second “shooting” mode corresponding to a path of lines of continuous or pseudo-continuous printing.

The performance of the robot in providing these two types of action—positioning and path—are very specific in terms of speed (up to 8 m/s) and precision (±20 μm). The weight moved by the robot is also an important criterion in terms of inertia. In general, the robot is used to transport cell culture dishes or multi-well dishes, which are very light objects that have no impact on the performance of the robot.

The link between the robot and the target is provided by an effector, which generally takes the form of a clamp.

### Implementation of Embodiments of the Present Disclosure

Moving the target through space in 3D and at three possible angles by means of the robotic arm opens the way to full compatibility with printing on nonplanar surfaces. Indeed, thanks to this approach, any printing point of the target can be placed in the same position with respect to a printhead, thus making it possible to maintain optimal printing conditions at all times. It should be emphasized that such an ability makes the solution compatible with in situ or even in vivo printing. However, a relative limitation should be noted in this context regarding the ability of the robot to move the target relative to the head as a function of the size and weight of the target. It can therefore be concluded that the performance and dimensions of the arm will have to be optimized with regard to the size of the printing medium to be moved.

This embodiment is particularly suitable for the manufacture of a curved biological tissue, for example, heart valves, corneas, blood vessels, cartilage deposited on a prosthesis, etc.

In particular, the effector of the robot can support a rotating cylindrical mandrel onto which the biological materials are transferred.

Another advantage lies in the ability to easily reload the printhead(s) with bio-ink because they are linked to the frame of the bioprinter. It is even easily conceivable to change the printheads or their reservoir without having to remove the printing support from the robot arm, making it possible to maintain the 3D positioning of the object to be printed even when it requires a large amount of raw material to print.

The robot also enables the target to be displaced relative to a plurality of printheads in order to alternate the bioprinting mode. For example, the robot can move the target relative to a laser pulse transfer head in order to deposit first series of biological materials—cells, for example—and then to an extrusion or ink jet printing nozzle in order to deposit particles of second series of biological materials—the extracellular matrix, for example.

Finally, the robot arm makes it possible to perform movements similar to those of the human hand, which opens the way to displacements of the receiving support along paths that ensure that the integrity of the shape of the printed object is preserved. Actually, in the field of bioprinting, printed materials have a certain flexibility, even more or less liquid parts. It is therefore necessary that the paths of displacement of the target be studied so as not to disturb the printed layers, which can be done by a robot arm that includes the 6 degrees of freedom necessary for this capacity.

Beyond the advantages associated with the specific implementation of the robot arm with respect to the target, the present disclosure proposes taking advantage of the automation of the printing processes through the contribution of the robotic arm. After all, the arm will make it possible to produce repeatable and precise prints while minimizing the manual operations by users of the bioprinter.

Thus, the arm is used:
in the phases upstream from printing: for the preparation of inks, pipetting, spreading of inks, filling of reservoirs, calibration, displacement of a cover, piercing of a septum, etc.
during the printing phases: for loading the target, moving the target relative to the printheads, printing path, unloading the target, removing the tip from a pipettor, activating the donor by an actuator, etc.
during the maturation phase: if the bioprinter is equipped with an incubator or connected to an incubator, the arm will be able to position the target inside it, carry out changes of media, bring the target to a characterization means (imaging type), etc.
during the conditioning phase: for placing the target tissue into a dedicated sterile envelope.

A non-limiting example of the preparation of the donor with the robotic arm consists in using an effector carrying a pipette (40) that is controlled via an actuator.

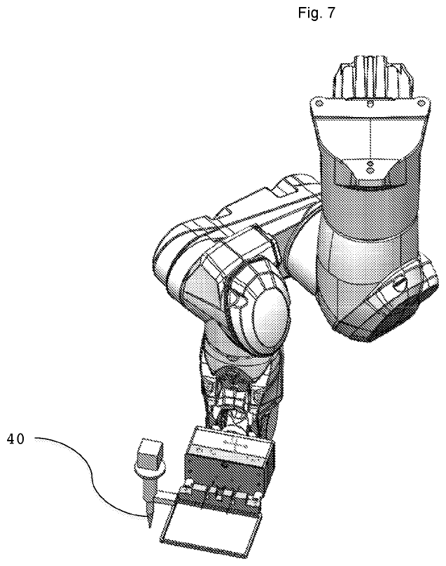

The robot arm first positions the pipette above the reservoir containing the ink. Then, the actuator makes it possible to perform a plurality of suction and ejection movements in order to mix and homogenize the ink. Then, the actuator makes it possible to take a sample of a controlled volume of ink, and the arm transports this volume from the area of the reservoir to the printhead, where it ejects the volume of ink taken from the donor. A particular case of this example consists in using a disposable cone between each preparation of the donor. It is essential to be able to minimize the time elapsed between the end of donor preparation and the start of laser printing. For this purpose, the effector of the robot carrying the recipient can carry the pipettor and actuator system, thus minimizing the displacement distances between the ink deposition system and the print recipient system. FIG. 7 illustrates this example.

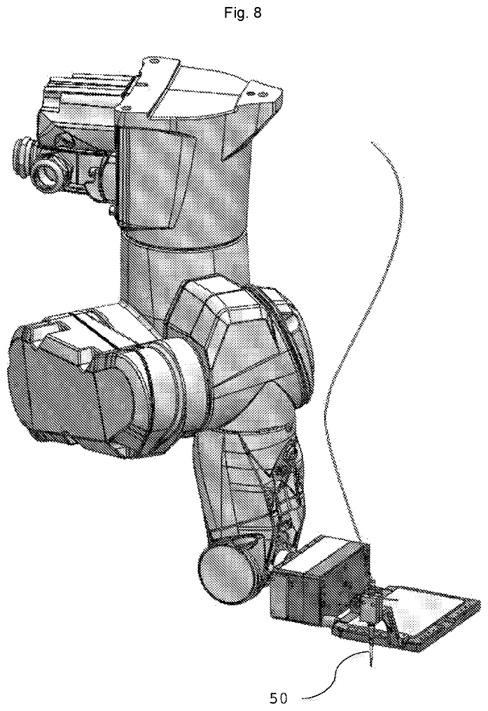

Another non-limiting example of donor preparation is based on the use of a pneumatic system. In this configuration, a positive- and negative-pressure controller enables the liquid to be expelled and suctioned, a solenoid valve system enables the pressure controller to be disconnected from the rest of the system, and a tube enables the preceding elements to be connected pneumatically to a sampling head, which can be a pipetting cone (50), for example. In this configuration, the volume taken “omega” can be controlled through the time “t” of pressurization “Delta P” according to the formula: omega=delta P/Rh*t, where Rh represents the hydrodynamic resistance. Unlike the previous system based on a pipette with actuator, precise control of the volume withdrawn is more difficult because the hydrodynamic resistance is strongly dependent on conditions such as the geometry of the reservoir or the position of the sampling cone therein. Thus, the reservoir containing the ink can be aliquoted beforehand with precise volumes (for example, 12 μl per well in a 384-well plate). When pipetting using the pneumatic system, even if too large a volume is taken, it will be composed of the predefined volume plus a volume of air that will play a benign role. In order to increase the sampling precision of this system, a control tool (monitoring the height of the sampled liquid, for example) and feedback loop (which adapts the pressures accordingly) can be set up. This example is illustrated in FIG. 8.

The robot can also perform a procedure to calibrate the position of the printheads in space. Indeed, printing by extrusion or microvalve requires perfect knowledge of the position of the printing needle relative to the surface of the recipient. As the recipient is carried by the robot, which positions it precisely with respect to these needles, it is possible to add a function of measuring the position of the needles on the robot. It can thus recalibrate the position of the hands at any time. The measuring means for carrying out this operation can be of different types such as, for example, an optical fork, a mechanical feeler, a camera, a laser beam, etc.

Given the link between robot arm and target, the printing paths will be provided by the robot arm itself, with the printheads remaining stationary. The printing time will therefore depend in part on the speed and precision of the robot, which are selected according to the intended application and the type of object to be printed. The print file will also be specific, since it is calculated with respect to the position of the target and no longer with respect to the position of the printheads as is the case in the prior art. In fact, the optimization of the printing path, which is strongly linked to the specifications of the robot and to the calculation of the printing pattern, is specific to the configuration described in the present disclosure. Thus, mathematical optimizations of the “traveling salesman” or machine learning type will make it possible to minimize printing time while ensuring that the desired pattern is obtained and that the previously printed layers are preserved (no sudden or excessively fast movements). The implementation of algorithms working in real time is necessary in order to ensure a short printing time that is compatible with the preservation of the cellular viability of the printed object. In this context, the use of a programmable logic controller will also allow for overall optimization of the printing through the real-time management of various sensors, of the robot arm, of the effector, of the printing heads, of the characterization means, etc. More generally, automation will directly serve the interests of medical bioprinting applications, since this field of automation/robotics is very highly standardized and thus makes it possible to simultaneously ensure performance, reproducibility, and safety, which are three essential requirements of the clinical sector.

The massive use of sensors and measurements in the enclosure where the robot is located will be necessary both in order to optimize printing along the way and to optimize future printing (paths, printing conditions, printing modes, etc.) through post-printing analysis. The latter will be based on developments in massive information processing (big data) and algorithms (machine learning, deep learning) that are widely used today. One can even imagine that artificial intelligence could be used to optimize the robotic bioprinting process, because it could make it possible to predict particular embodiments.

It will be readily understood that the connection to the outside of such a bioprinter, particularly to databases, will make it possible to instrument and monitor all the prints, thus enabling enormous gains to be made in the capacity of such a bioprinter to deliver tissues that fully meet the objective at the application level.

The present solution is universal in the sense that the printing mode, whether oriented upward or downward, is compatible with the use of a robotic arm capable of rotating the target 360°. The printing of cells by laser upward and the printing of biomaterials downward by extrusion or microvalve can thus be used jointly within the same bioprinter thanks to the contribution of the 6-axis robot arm, thus taking advantage of the best known configurations of each printing method.

According to one variant, it would be possible to integrate a plurality of robot arms: For example, a first robot arm could be devoted to pre-printing operations, another to printing, and finally a last to the post-printing phase. In this context, there would be no more manual operations on the part of users. Different multi-robot configurations are possible in this context. The robot arm can also be associated with other automated or manual conveying means, whether they form part of the enclosure or not.

According to another variant, the robot arm could transport a plurality of targets via one or more effectors in order to parallelize the prints with respect to a plurality of stationary printheads. This type of configuration is advantageous when bioprinting requires high throughputs in volume or in number of tissues to be manufactured, particularly in a production mode.

According to another variant, the robot arm (via its effector) could include active functions such as lighting, imaging, heating, position sensors, etc., in order to instrument the target so as to allow for:
a longer print,
calibration,
collection of target-specific data during printing,
direct characterization of what is printed during the printing phase (inline measurement).

According to one variant, the robot arm is GMP compatible (requirements of the pharmaceutical sector) in order to allow for the manufacture of clinical-grade tissues.

According to one embodiment, the system comprises a station for acquiring a digital model of the target consisting of a camera that produces a series of images of the target being displaced by the robot.

According to another variant, the system comprises one or more cameras analyzing the target, particularly a living or deformable target, in order to recalculate the position of a zone of interest intended to receive the transfer of the biological material into the robot's frame of reference, the robot recalculating the path in real time according to the configuration of the target.

For an extrusion manufacturing mode, the robot positions a position sensor in front of an extrusion head during an initialization phase in order to precisely calibrate the position of the distal plane of the extrusion orifice of the nozzle.

According to another variant, the system comprises human interaction means for ensuring the displacement of the target, and robotic means for controlling the displacement of the robot. This variant makes it possible, in particular, to carry out training of the displacements or of the slaving of the displacements by a human action supplemented by the action of the robot.

According to a particular mode of operation, the robot controls the rotation of its effector in order to ensure the spreading of the bio-ink film in the context of laser printing.

According to other variants, the system is controlled by a computer executing a program for controlling the joints of the robot according to an algorithm for optimizing the path. For this purpose, it comprises sensors for detecting the position of the robot and, for example, learning processes for determining the optimal paths.

The system is designed to allow sterilization in order to enable direct implementation in a surgical block.

According to another variant, the printing means—e.g., laser—is located in the same space as the robot and the target. In this case, the printing medium should be designed to minimize particle emissions so as not to interfere with the printing process. This scenario corresponds to a situation in which the entire bioprinting system is implemented in a single space, which can be an enclosure that can be opened, a closed enclosure, or even a room dedicated to bioprinting.

1. A bioprinting system for the manufacture of a structured biological material from materials at least part of which consists of living biological particles (cells and cellular derivatives), comprising:
a) a printing assembly containing at least one printhead for objects of biological interest and at least one target,
b) a source for supplying the printhead with objects of biological interest,
c) a means for bioprinting the objects of biological interest, and
d) a displacement means for relative displacement of the printhead with respect to the target, the displacement means comprising a robot configured to control the displacement of the target along six axes, and
wherein at least one of the printing heads is stationary during printing.

2. The bioprinting system of claim 1, wherein the robot is a robotic arm having six degrees of freedom, with three axes intended for positioning and three axes for orientation along at least 180° for each axis of rotation, making it possible to displace and orient the target in a given workspace, the course of the displacements being greater than the largest dimension of the target.

3. The bioprinting system of claim 1, wherein the robot is a hexapod robot.

4. The bioprinting system of claim 1, wherein the robot is a delta robot.

5. The bioprinting system of claim 1, wherein the robot is a hexapod robot or a delta robot and comprises a means for turning the target.

6. The bioprinting system of claim 1, wherein the target is linked to the robot by an effector.

7. The bioprinting system of claim 1, further comprising a support for receiving a plurality of targets, the robot configured to control the extraction of a target for displacement relative to the bioprinting means.

8. The bioprinting system of claim 1, further comprising a second robot for performing an additional function (pipetting, etc.).

9. The bioprinting method of claim 1, wherein the bioprinting system incorporates at least one laser bioprinting device.

10. The bioprinting system of claim 1, wherein the bioprinting system incorporates at least one nozzle bioprinting device.

11. The bioprinting system of claim 10, wherein the bioprinting system integrates a combination of a nozzle and a laser bioprinting device.

12. A bioprinting method for the manufacture of a structured biological material from materials at least a portion of which comprises biological particles comprising controlling a displacement of at least one target by means of a robot in three dimensions relative to at least one stationary printhead during printing of the materials.

13. The bioprinting method of claim 12, further comprising displacement of the target relative to at least one workstation.

14. The bioprinting method of claim 13, wherein the displacement is controlled in order to maintain a constant distance between a target having a nonplanar surface, and a printhead.
